(19) United States
(12) Patent Application Publication
(10) Pub. No.: US 2018/0344210 A1
(43) Pub. Date: Dec. 6, 2018

(54) Extensible Wearable Weight Scale and Sensor System

(71) Applicant: Weighday, LLC, Arlington, TX (US)

(72) Inventor: Marcus G. Pestl, Dallas, TX (US)

(21) Appl. No.: 15/610,155

(22) Filed: May 31, 2017

(51) Int. Cl. A61B 5/103; A43B 3/00; A43B 17/03; A43B 17/02; A61B 5/00; A61B 5/053

(52) U.S. Cl. CPC A61B 5/103; A43B 3/0005; A43B 17/035; A43B 17/026; A61B 5/6802; A61B 2560/0214; A61B 5/742; A61B 5/4872; A61B 5/4875; A61B 2562/0247; A61B 5/053

(57) ABSTRACT

A wearable weight scale, including an extensible fluid-filled insole for measuring and producing data relating to pressure changes within the extensible fluid-filled insole; a transceiver disposed about the extensible fluid-filled insole for transmitting the data; and one or more electronic devices for wirelessly receiving the transmitted data for displaying the data to a user.

### FIELD OF THE DISCLOSURE

This invention relates, in general, to an extensible wearable weight scale, and in particular, to an extensible wearable weight scale and sensor system.

### BACKGROUND

Without limiting the scope of the present disclosure, its background will be described in relation to an extensible wearable weight scale and sensor system, as an example.

The commercial advantages of a personal weighing device incorporated as part of footwear are manifold. There has been no successful commercialization of an insole that is a personal weighing device that combines an extremely lightweight configuration, and that can also be folded up into a small article that fits easily in a purse, bag, and the like would therefore have distinct advantages.

For decades, conventional personal weighing scales have consisted of a single, rigid plate—nowadays often metal and plastic or glass—that is supported by, and transfers the user's weight to, four force-measuring load-cells, one located approximately at each corner of the underside of the rigid plate. Four force-measuring cells on a rigid plate are required so a person can stand comfortably on the scale plate while at the same time not transferring any of their weight to the ground where it would not be measured by the load-cells. The measurement of static force from these four load-cells is aggregated in analog form to a total reading of the user's weight, and manufacturers of such scales generally claim these products are accurate within a range of plus or minus two to three percent. There are many versions of these conventional scales available for sale in large and smaller sizes; however, the key disadvantage of the smaller style versions is that the rigid plate is too small to stand on without both feet overhanging the plate. In addition, these items are too heavy to be considered truly portable and could easily break or be damaged if being carried in a suitcase or handbag. Further, certain scales cannot fold into a smaller form. Some scales have a limited weight capacity and/or only a single force-measuring cell located centrally when the scale is unfolded so it can only be used as a kitchen scale. It cannot weigh a person, because the platform where the goods to be weighed must be placed is no larger than a few inches in diameter, and therefore could not possibly accommodate a person's feet for the purposes of determining their weight. If attempting to weigh themselves on this scale, a person would additionally have to balance on this single, force-measuring sensor without transferring any of their weight to the surrounding supporting structure, which would be a physical impossibility, because an average person's feet comprise several dozen square inches of area, and the average person cannot balance on an area the size of a jelly jar lid for the purposes of weighing themselves. Even if the plate size could be expanded to accommodate a person's feet, this plate would have to be foldable to fit in a purse or pocket, and also rigid enough to support up to 350 to 400 pounds of weight—the current consumer expectation of the weight capacity of a personal scale. Yet no such embodiment of an insole insertable into footwear exists as prior art. Moreover, even if such a folding plate that supported 350 to 400 pounds existed, there is no teaching to locate additional force-measuring cells away from the center of the plate in order to provide a firm supportive surface for a person to stand on and transfer all of their weight to these additional force-measuring cells.

A further development in personal weighing scales has been the addition of various biometric sensors in the surface of the plate where one's feet are placed so that the functionality of the device is enhanced beyond simply providing a measure of one's weight. The purpose of these biometric sensors is to provide measurements such as the user's body water and body fat content by transmitting an electrical signal through the user's body and measuring the current thereof to provide a value that expresses body water and body fat as a percentage of the user's weight. As the most commercially cost-effective versions of these sensors are rigid metal strips that cannot be folded and must remain flat, a reduction to practice of a lightweight, foldaway body weight analysis device requires these sensors be mounted on areas of the device that remain rigid and flat when the device is folded. Furthermore, these sensors must be connected by wire, or wirelessly, to a display attached to, or in communication with, the device that converts sensor input to information capable of being understood visually or aurally, or by some other output, to a human user or by remote data processing means.

### SUMMARY

Embodiments of the present disclosure are directed to an extensible wearable weight scale and sensor system. In one embodiment, the present disclosure is directed to an extensible wearable weight scale sensor system, including a fluid-filled insole for measuring and producing data relating to pressure changes; a transceiver disposed about the fluid-filled insole for transmitting the data; and one or more electronic devices for wirelessly receiving the transmitted data for displaying the data to a user. In one aspect, the fluid-filled insole may include at least one pressure sensor for measuring pressure of at least one of a gas and a fluid in a lower portion bladder disposed within the fluid-filled insole.

In another aspect, the fluid-filled insole may include at least one bio-impedance sensor for measuring electrical signals transmitted through the body of the user. Also, the at least one bio-impedance sensor may be disposed about a metalized fabric upper portion substantially disposed over the lower portion bladder. Additionally, the fluid-filled insole may further include one or more structural members in structural communication between an upper surface and lower surface of the fluid-filled insole for providing resistance and structural profile consistency of the fluid-filled insole when under pressure.

In yet another aspect, the fluid-filled insole may include an electronic module in communication with the at least one pressure sensor and transceiver for producing the data. Further, the fluid-filled insole may include a charging pump for adjusting pressure within the fluid-filled insole.

In another embodiment, the present disclosure may be directed to a fluid-filled insole, including a flexible upper portion having at least one bio-impedance sensor disposed about its upper surface; an inflatable, flexible lower portion substantially disposed under the flexible upper portion; and an electronic module disposed about the inflatable, flexible lower portion for wirelessly receiving instructions from and transmitting data to at least one electronic device. In yet another embodiment, the fluid-filled insole may include a power source, a transceiver, a memory, and a digital processor.

In still yet another aspect, the fluid-filled insole may include at least one pressure sensor for measuring pressure of at least one of a gas and a fluid in the lower portion. Also, the fluid-filled insole may further include one or more structural members in structural communication between an upper surface and a lower surface of the extensible liquid-filled insole for providing resistance and structural consistency of the fluid-filled insole when under pressure. Additionally, the fluid-filled insole may include an electronic module in communication with the at least one pressure sensor and transceiver for producing the data.

In still yet another aspect, the fluid-filled insole may include a charging pump for adjusting pressure within the fluid-filled insole. In addition, the fluid-filled insole may include a release valve for adjusting pressure within the fluid-filled insole.

In yet another embodiment, the present disclosure may be directed to a method for displaying footwear data on an electronic device, the method including measuring an initial pressure of at least one of a gas and a fluid bladder disposed within the footwear; subsequent to placing a load on the footwear by a foot of a user, measuring the resultant load pressure; calculating the pressure differential between the initial pressure and the resultant load pressure; converting the pressure differential into a weight measurement; and transmitting at least one of the pressure differential and the weight measurement to an electronic device.

In one aspect, the measuring an initial pressure may include prior to insertion of the foot into the footwear, converting electrical signals of one or more pressure sensors into an initial weight measurement. In another aspect, after insertion of the foot into the footwear, electrical signals of one or more pressure sensors may be converted into a resultant weight measurement. Also, the method may include providing substantially consistent structural thickness of the at least one of a gas and a fluid bladder by intermittent structural members in communication with the upper and lower surface of the at least one of a gas and a fluid bladder.

In addition, the method may include increasing the pressure within the at least one of a gas and a fluid bladder disposed within the at least one of a gas and a fluid bladder. Further, the method may include decreasing the pressure within the at least one of a gas and a fluid bladder disposed within the at least one of a gas and a fluid bladder. The method may further include measuring at least one of body fat percentage, body water percentage, and steps counted.

### BRIEF DESCRIPTION OF THE DRAWINGS

For a more complete understanding of the features and advantages of the present disclosure, reference is now made to the detailed description along with the accompanying figures in which corresponding numerals in the different figures refer to corresponding parts and in which:

FIG. 1 is an illustration of an extensible wearable weight scale and sensor system according to an embodiment of the present disclosure;

FIG. 2 is a side cross sectional view of a footwear having an extensible wearable weight scale according to an embodiment of the present disclosure;

FIG. 3 is a top view of the extensible wearable weight scale of FIG. 2 according to an embodiment of the present disclosure;

FIG. 4 is a partial side cross sectional view of the extensible wearable weight scale of FIG. 2 according to an embodiment of the present disclosure;

FIG. 5 is a schematic diagram of the extensible wearable weight scale and sensor system according to an embodiment of the present disclosure;

FIG. 6 is a schematic diagram of an electronic module of FIG. 5 according to an embodiment of the present disclosure;

FIG. 7 is a side view of an extensible wearable weight scale that is rolled together according to an embodiment of the present disclosure;

FIG. 8 is a series of graphical user interface displays on a user's electronic device according to an embodiment of the present disclosure;

FIG. 9 is a graphical representation of data obtained from one embodiment of the extensible wearable weight scale and sensor system;

FIG. 10 is a flowchart of a method for displaying data to a user according to an embodiment of the present disclosure; and

FIG. 11 is a flowchart of a method for displaying data to a user according to another embodiment of the present disclosure.

### DETAILED DESCRIPTION

While the making and using of various embodiments of the present disclosure are discussed in detail below, it should be appreciated that the present disclosure may provide many applicable inventive concepts, which can be embodied in a wide variety of specific contexts. The specific embodiments discussed herein are merely illustrative and do not limit the scope of the present disclosure.

Described herein are embodiments for an extensible wearable weight scale and sensor system (hereinafter “wearable weight scale system”), weight scale footwear, and methods of using the same.

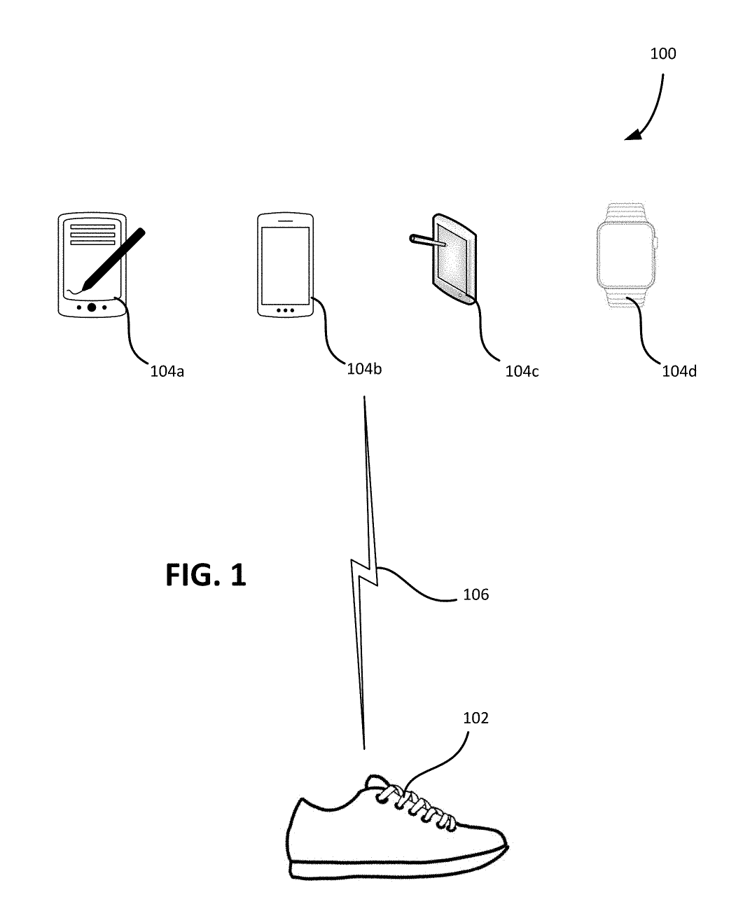

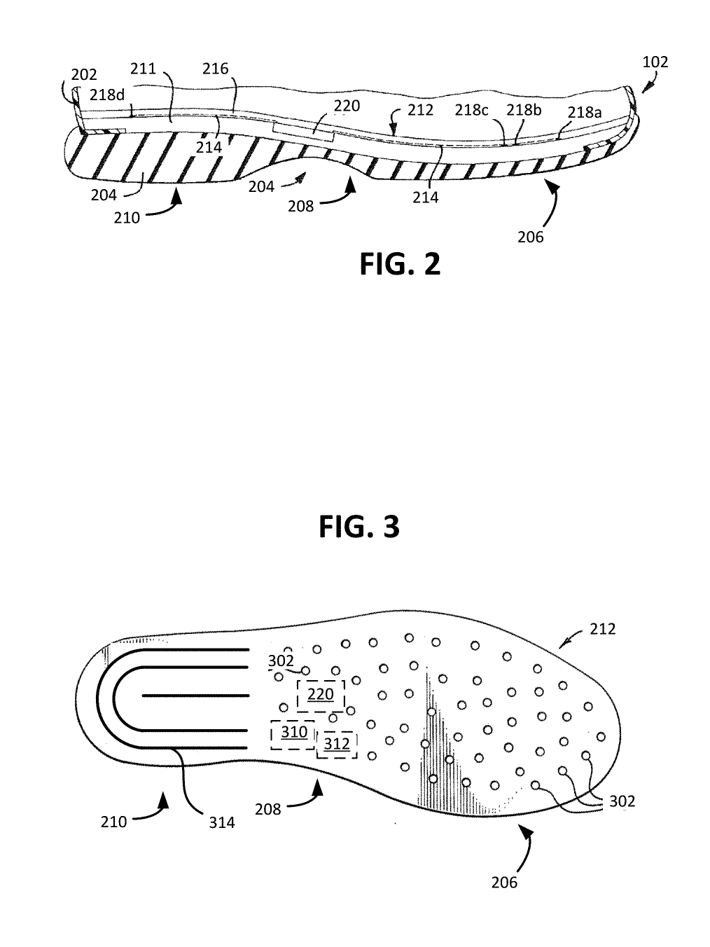

Referring initially to FIG. 1, wearable weight scale system 100 may include footwear 102, such as a shoe, boot, athletic shoe, insole, and the like. Although the example described refers to a shoe, footwear 102 may be any type of known footwear to those skilled in the art. As shown better with respect to FIG. 2, footwear 102 may include a separate or integrated liquid-filled insole as further described herein. Additionally, wearable weight scale system 100 may include one or more electronic devices, such as a tablet 104a, mobile device (smartphone 104b), portable computing device 104c, and smartwatch 104d. Collectively these devices may be referred hereinafter as electronic devices 104. Wearable weight scale system 100 may further include wireless communication link 106, such as an infrared link, radio frequency link, Bluetooth link, and/or any other communication link as are known to those skilled in the art.

With reference to FIG. 2, footwear 102 may include upper 202 and sole structure 204. In general, footwear 102 may be divided into three general sections: forefoot section 206, midfoot section 208, and heel section 210. Sections 206-210 are intended to represent general areas of footwear 102 that may provide a frame of reference. Although sections 206-210 apply generally to footwear 102, references to sections 206-210 also may apply specifically to upper 202, sole structure 204, or individual components included within and/or formed as part of either upper 202 or sole structure 204.

Upper 202 may be secured to sole structure 204 and define a void or chamber for enclosing a foot of a user. Footwear 102 may include any desired closure mechanisms known to those skilled in the art and utilized in any manner that secures the foot of a user within footwear 102.

Sole structure 204 may be secured to a lower surface of upper 202 and may have a generally conventional shape. Sole structure 204 may be a multi-piece structure including any of forefoot section 206, midfoot section 208, and heel section 210. Additionally, footwear 102 may include integrated or removable insole member 212 as further discussed below. Additional support structure 211 may be located between insole member 212 and sole structure 204, in one aspect of the present disclosure. Insole member 212 may or may not directly contact the foot of a user, as another element may interfere with direct contact, such as a sock and the like. In such an article, the upper portion of the sole structure may be considered a foot-contacting member, even though it may not directly contact the foot of the user.

Insole member 212 may include inflatable lower portion 214 and metalized upper portion 216 as further described herein. In one embodiment, lower portion 214 may include one or more pressure sensors 218a-218d (collectively pressure sensors 218). Additionally, lower portion 214 may include electronic module 220 that may also include pressure sensor 218. In one embodiment, insole member 212 may contain any number of pressure sensors 218. In another embodiment, insole member 212 may contain a single pressure sensor 218 that may be located in electronic module 220. Pressure sensors 218 may be located in insole member 212 where pressure points of a foot contacting an insole would be known, such around the balls and heel of a user's foot.

In one embodiment, if pressure sensors 218 are located in other parts or regions of insole member 212 away from electronic module 220, then they may be in communication with electronic module 220 via wired and/or wireless communication links, such as wire leads, conductors, and the like.

In one embodiment, footwear 102 may include insertable insole member 212, and in another embodiment, footwear 102 may have incorporated into the manufactured structure the elements and functions of insole member 212, thereby not requiring an insertable insole member.

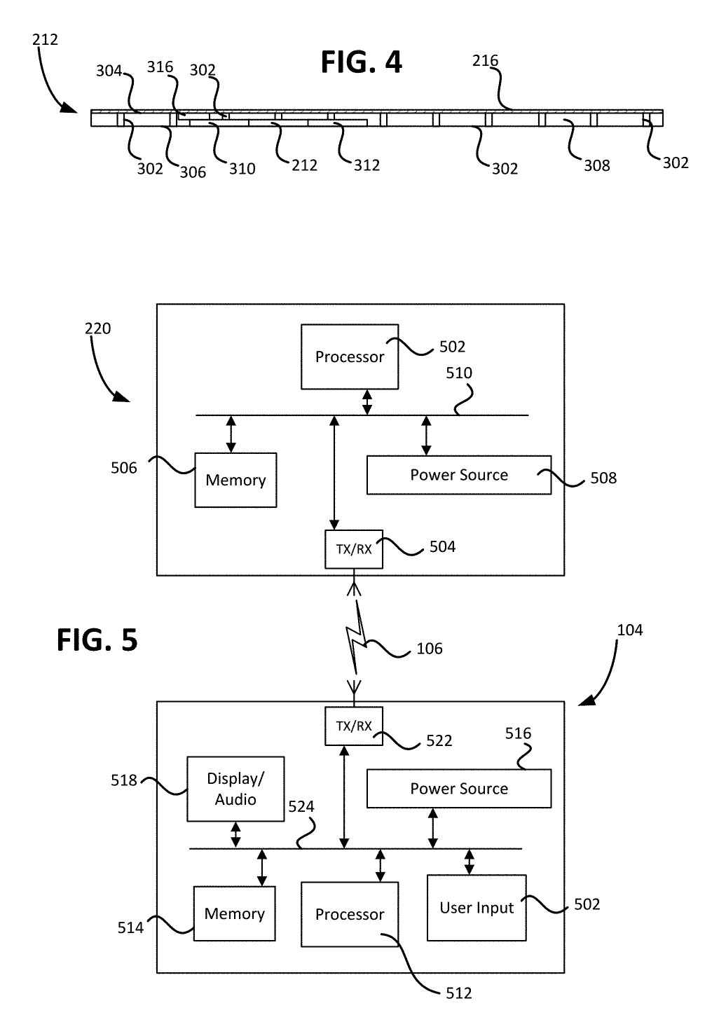

With reference to FIGS. 3 and 4, an embodiment of insole member 212 is shown. Insole member 212 may include one or more structural members 302 that provide structural rigidity for upper surface 304 and lower surface 306 of lower portion 214 of insole member 212. Structural members 302 may provide resistance to the pressure exerted by a gas, liquid and/or fluid (hereinafter referred to as a “fluid”) contained within bladder 308 of lower portion 214 of insole member 212 such that lower portion 214 of insole member 212 may be presented with a substantially uniform thickness when pressurized within footwear 102.

In one embodiment insole member 212 may be a flat device that provides a large enough surface area to support the average person's feet when standing, and their weight, yet can be removed from footwear 102 and folded or rolled up in such a way that it becomes smaller in at least one dimension, as best shown in FIG. 7, so it can fit in a person's purse or pocket.

In one embodiment, insole member 212 may be constructed with a plurality of exposed rigid or flexible conductive plates or strips fixed to upper surface 216 of insole member 212 that measure the resistance of a current passed through the user's body to provide sensor inputs that measure biometric data, such as body fat percentage, and means to convert said sensor inputs into information that can be readily interpreted by a human user or by remote data processing means.

In one embodiment, lower portion 214 of insole member 212 may be equipped with bladder 308 constructed from material, such as flexible thermoplastic, that is shaped into sealed tubular or bladder-type structures that can be filled or inflated with any of a gas, liquid and/or fluid, including but not limited to, air. Bladder 308 may be linked to electronic module 220 that accurately captures a person's weight by means of measuring pressure differential when a person's weight is applied to bladder 308. It should be noted that while the embodiment shown describes bladder 308 formed from sheets of material, bladder 308 may be constructed from any tube or structure that can contain a volume of pressurized gas or fluid, and thus embodiments of the present disclosure are not limited to the embodiment as shown and described herein. Additionally, insole member 212 may include charging pump 310 and release valve 312 for pressurizing bladder 308 of insole member 212 to a desired pressure. Pump 310 and release valve 312 may be accessible and operable along an outside surface of insole member 212.

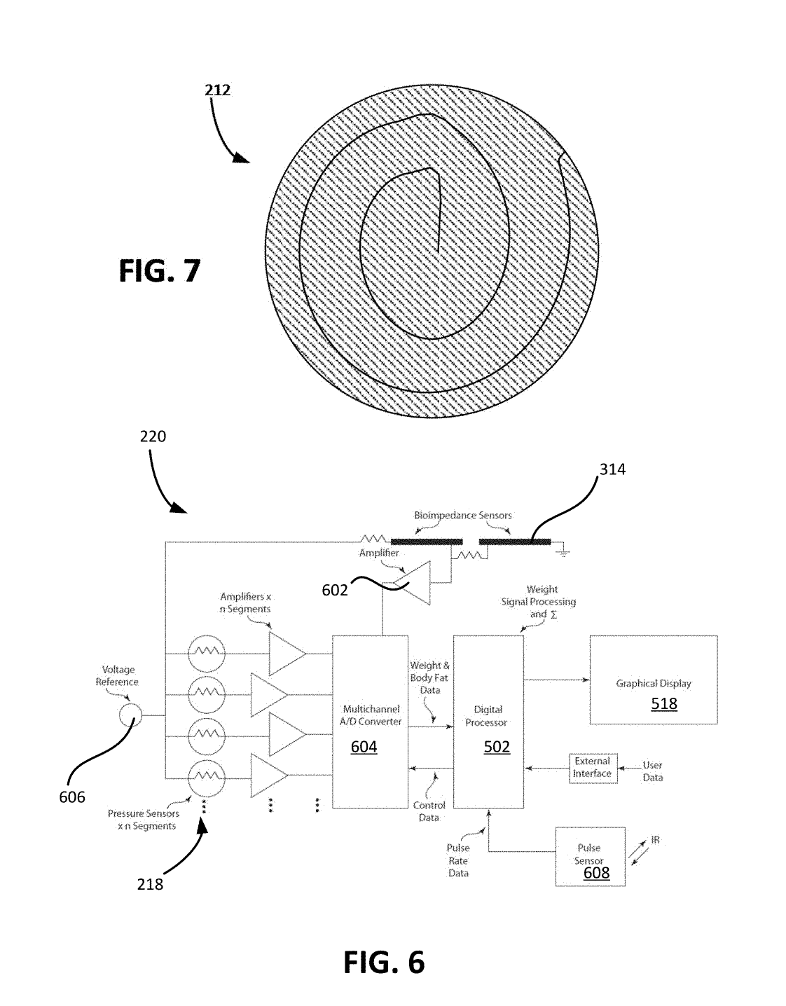

In one embodiment, one or more of lower portion 214 and upper portion 216 may include bio-impedance strips 314, in the form of stainless steel or flexible metallized fabric strips illustrated in FIG. 3, biased using a fixed voltage reference 606 as shown in FIG. 6. In one embodiment, bio-impedance strips 314 are disposed about heel section 210 of the upper surface of upper portion 216 of insole member 212.

With reference to FIG. 5, electronic module 220 may include digital processor 502 for processing electrical signals produced by any of the components/elements of footwear 102. Additionally, electronic module 220 may include data transceiver/receiver (TX/RX) component 504 for transmitting data to and/or receiving data from one or more electronic devices 104. Also, electronic module 220 may include memory system 506, and power source 508 (e.g., a battery or other power source). A bus, computer architecture pathway, (such as bus 510) and the like may be provided for all elements/components of electronic module 220 to communicate with each other and electronic devices 104.

Connection to one or more pressure sensors 218 can be accomplished through TX/RX 504, but additional sensors (not shown) may be provided to sense or provide data or information relating to a wide variety of different types of parameters, such as physical or physiological data associated with use of the article of footwear 102 or the user, including, but not limited to, pedometer type speed and/or distance information, other speed and/or distance data sensor information, temperature, altitude, barometric pressure, humidity, GPS data, accelerometer output or data, heart rate, pulse rate, blood pressure, body temperature, EKG data, EEG data, data regarding angular orientation and changes in angular orientation (such as a gyroscope-based sensor), etc., and this data may be stored in memory system 506 and/or made available, for example, for transmission by TX/RX 504 to some remote location or system.

Additional sensor(s), if present, may also include accelerometers (e.g., for sensing direction changes during steps, such as for pedometer type speed and/or distance information, for sensing jump height, etc.).

As discussed above, electronic module 220 may be configured to communicate with electronic devices 104, which may be an external computer or computer system, mobile device, gaming system, or other type of electronic device, as described previously. Electronic devices 104 may include processor 512, memory 514, power supply 516, display 518, user input 520, and TX/RX 522. TX/RX 522 may be configured for communication with electronic module 220 via TX/RX 504 of electronic module 220, through any type of known electronic communication, including the contacted and contactless communication methods described above and elsewhere herein.

It is understood that electronic module 220 can be configured for communication with a plurality of external devices, including a wide variety of different types and configurations of electronic devices. Additionally, TX/RX 504 of electronic module 220 may be configured for a plurality of different types of electronic communication. It may be further understood that footwear 102 may include a separate power source to operate pressure sensors 218, if necessary, such as a battery, piezoelectric, solar power supplies, or others. Pressure sensors 218 may also simply receive power through connection to electronic module 220.

With reference to FIG. 6, information and/or data from bio-impedance strips 314 may be amplified via amplifier 602 and fed into a high resolution multichannel A/D converter 604 and digitized. The digitized information from bio-impedance strips 314 is in turn fed into processor 502 of electronic module 220, and utilizing Bioelectrical Impedance Analysis techniques, a variety of biometric data may be calculated, including but not limited to, body fat, hydration levels, and muscle density. Further, footwear 102 may include a charging port, such as USB charging port 316 for connecting with an external power source for charging power source 508 (FIG. 5).

Key variables required for this calculation may be stored in processor 502, and input to processor 502 via an external digital interface. The resultant computed biometric data may be displayed on display 518. User-specific biometric values and weight collected by the device may also be transmitted wirelessly to any other device or information repository, and the display of such information should not be considered as limited to the embodiment shown and described herein.

Further, electronic module 220 may include an infrared pulse (IR) sensor 608. For example, a reflective IR-based pulse sensor utilizing a high-intensity IR LED and IR sensor may also be embedded within a portion of bio-impedance strips 314. Variation in the reflected infrared from the high-intensity IR LED may be received by the IR sensor and may correspond to heart rate. Utilizing AGC (automatic gain control) circuitry and a threshold detector, information from the IR Sensor may be converted into the form of a digital pulse train, corresponding to a pulse. This may then be fed into a digital processor where, utilizing a time base internal to the digital processor, the digital pulse train may be converted into heart rate data in the form of BPM (Beats Per Minute) and also displayed on display 518. User-specific heart rate values collected by the device may also be transmitted wirelessly to any other device or information repository, and the display of such information should not be considered as limited to the embodiment shown and described herein.

Additionally, footwear 102 may provide additional data to electronic devices 104, such as body fat percentage, body water percentage, and steps counted. FIG. 7 is an illustration of an embodiment of insole member 212 being folded or rolled together to show its compactness relative to prior art devices.

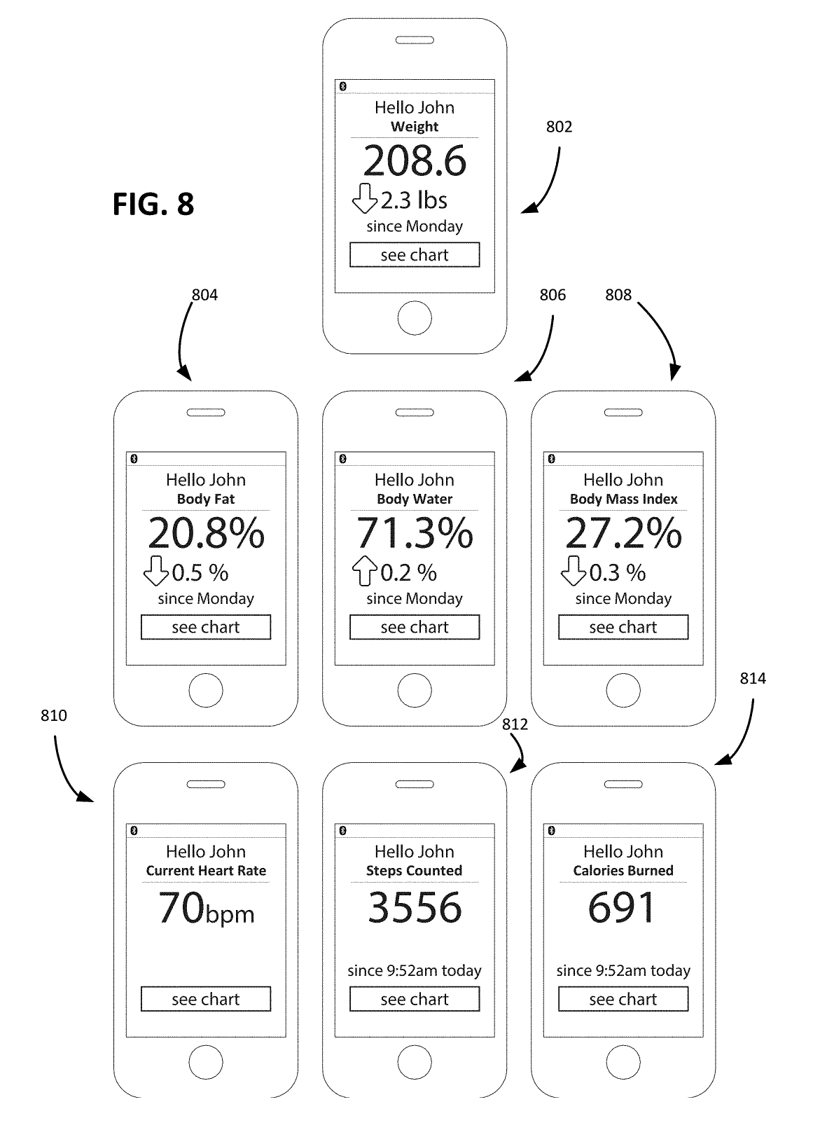

FIG. 8 depicts a series of graphical user interface (GUI) displays 802-814 (collectively GUIs 800). GUI 802 may display to a user on one or more of electronic devices 104 a welcome to the user's name and displaying a weight delta as computed as herein described. It may also note a reference date for the base calculation, such as a day before, a few days before, months before, etc. It may also display additional charted information. The term “delta” may mean a previous measurement of a biological data point such as weight differences from one calendar date to another date, for example.

GUI 804 may display to a similar welcome to the user and body fat delta as computed as herein described. It also may display additional charted information as shown. GUI 806 may display to a similar welcome to the user and body water delta as computed as herein described. It also may display additional charted information as shown. GUI 808 may display to a similar welcome to the user and body mass index delta as computed as herein described. It also may display additional charted information as shown. GUI 810 may display to a similar welcome to the user and current heart rate as computed as herein described. It also may display additional charted information as shown. GUI 812 may display to a similar welcome to the user and steps counted as computed as herein described. It also may display additional charted information as shown. GUI 814 may display to a similar welcome to the user and calories burned as computed as herein described. It also may display additional charted information as shown.

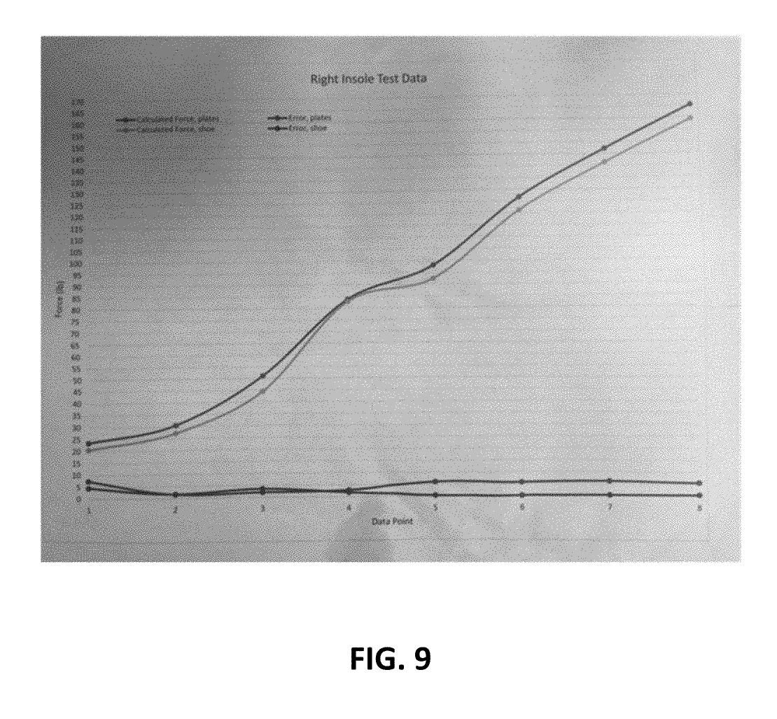

Turning now to FIG. 9, data collected relative to a load on footwear 102 in communication with electronic devices 104 may be compared to the actual testing load presented to footwear 102. The testing load readings may be compared with the readings displayed on electronic devices 104 and then may be graphically displayed and shown in FIG. 9. This data shows the accuracy of wearable weight scale system 100 in displaying the actual loads and data associated with wearable weight scale system 100.

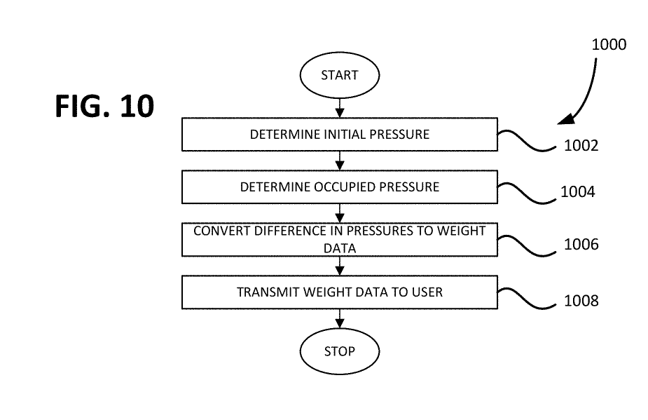

Referring now to FIG. 10, an embodiment of a method series of indicia 1000 for transmitting weight data to a user is described. In step 1002, an initial pressure reading of bladder 308 may be determined. In step 1004, a foot of a user may be inserted into footwear 102, and then another pressure reading of bladder 308 may be determined. In step 1006, the difference in pressures may be determined and either transmitted directly to electronic devices 104 for converting to a weight measurement, or may be converted directly and then transmitted to electronic devices 104 for displaying to a user as shown in step 1008.

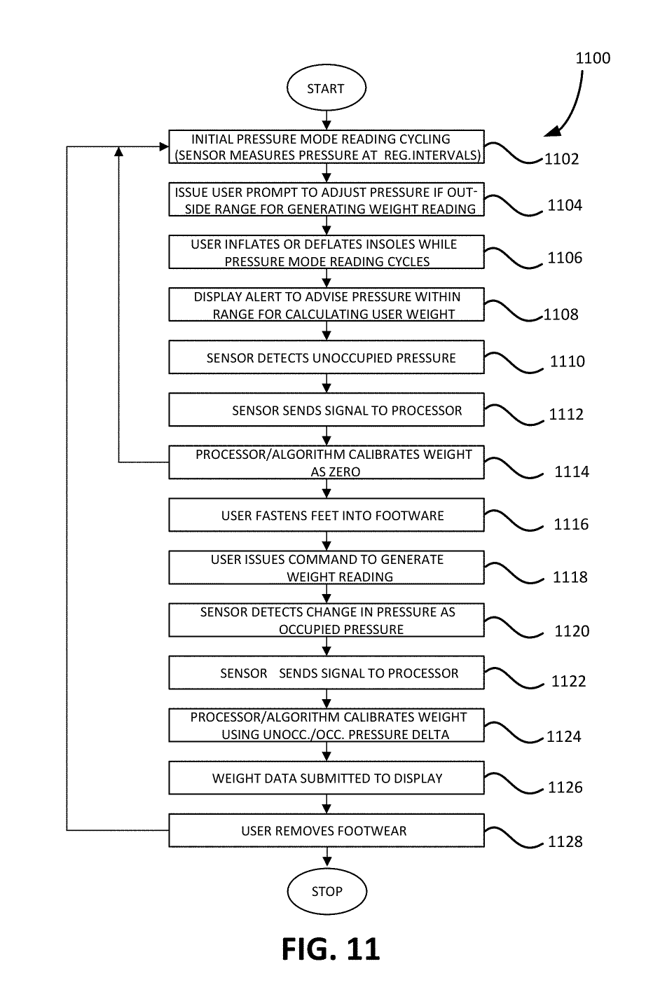

Turning now to FIG. 11, method 1100 for transmitting weight data to a user according to an embodiment of the present disclosure is described. In step 1102, the sensor may cycle at predetermined intervals to measure the current pressure of liquid or gas within bladder 308. In step 1104, if the pressure within bladder 308 as measured by the sensor is outside a predetermined range required to produce a weight reading, a user prompt may be issued to display 518 to instruct the user to inflate or deflate each insole as needed. In step 1106, the user may inflate or deflate bladder 308 while the user prompt may dynamically inform the user once the pressure is within range to produce a weight reading. In step 1110, and as in step 1102, sensor cycling may determine the current pressure of liquid or gas within bladder 308.

In step 1112, if processor 502 detects pressure within bladder 308 is within a sub-range limit, a processing algorithm may determine whether insole member 212 is unoccupied. In step 1114, if the insole member is unoccupied, the processing algorithm may calibrate the weight on the insole as zero. In step 1116, a foot of a user may be inserted into footwear 102. In step 1118, the user may issue a command to generate a weight reading by using a device in communication with electronic module 220. In step 1120, when a command is issued, sensor 218 may determine a pressure reading of bladder 308.

In step 1122, the difference value in pressure between unoccupied and occupied insole member 212 may be determined and sent electronically to processor 502. In step 1124, the difference value may be converted directly by processor 502 to a weight measurement value, or may be transmitted directly to electronic devices 104 for converting by processor 512. In step 1126, the weight data measurement may be displayed to a user on electronic devices 104. In step 1128, the user may remove the footwear.

While embodiments of the present disclosure have been described with reference to illustrative embodiments, this description is not intended to be construed in a limiting sense. Various modifications and combinations of the illustrative embodiments as well as other embodiments of the invention will be apparent to persons skilled in the art upon reference to the description. It is, therefore, intended that the appended claims encompass any such modifications or embodiments.

1. An extensible wearable weight scale sensor system, comprising:
a fluid-filled insole for measuring and producing data relating to pressure changes;
a transceiver disposed about the fluid-filled insole for transmitting the data; and
one or more electronic devices for wirelessly receiving the transmitted data for display to a user.

2. The extensible wearable weight scale sensor system of claim 1, wherein the fluid-filled insole comprises:
at least one pressure sensor for measuring pressure of at least one of a gas and a fluid in a lower portion bladder disposed within the fluid-filled insole.

3. The extensible wearable weight scale sensor system of claim 1, wherein the fluid-filled insole comprises:
at least one bio-impedance sensor for measuring electrical signals transmitted through a body of the user.

4. The extensible wearable weight scale sensor system of claim 3, wherein the at least one bio-impedance sensor is disposed about a metalized fabric upper portion substantially disposed over a lower portion bladder.

5. The extensible wearable weight scale sensor system of claim 1, wherein the fluid-filled insole comprises:
one or more structural members in structural communication between an upper surface and a lower surface of the fluid-filled insole for providing resistance and structural profile consistency of the fluid-filled insole when under pressure.

6. The extensible wearable weight scale sensor system of claim 2, wherein the fluid-filled insole further comprises:
an electronic module in communication with the at least one pressure sensor and the transceiver.

7. The extensible wearable weight scale sensor system of claim 1, wherein the fluid-filled insole further comprises:
a charging pump for adjusting pressure within the fluid-filled insole.

8. The extensible wearable weight scale sensor system of claim 7, wherein the fluid-filled insole further comprises:
a release valve for adjusting pressure within the fluid-filled insole.

9. A fluid-filled insole, comprising:
a flexible upper portion having at least one bio-impedance sensor disposed about an upper surface of the flexible upper portion;
an inflatable, flexible lower portion substantially disposed under the flexible upper portion; and
an electronic module disposed about the inflatable, flexible lower portion for wirelessly receiving instructions from and transmitting data to at least one electronic device.

10. The fluid-filled insole of claim 9, further comprising:
a power source, a transceiver, a memory, and a digital processor.

11. The fluid-filled insole of claim 9, further comprising:
at least one pressure sensor for measuring pressure of at least one of a gas and a fluid in the inflatable, flexible lower portion.

12. The fluid-filled insole of claim 9, further comprising:
one or more structural members in structural communication between an upper surface and a lower surface of the inflatable, flexible lower portion for providing resistance and structural consistency of the fluid-filled insole when under pressure.

13. The fluid-filled insole of claim 11, further comprising:
an electronic module in communication with the at least one pressure sensor and the transceiver.

14. The fluid-filled insole of claim 9, further comprising:
a charging pump and a release valve for adjusting pressure within the fluid-filled insole.

15. A method for displaying footwear data on an electronic device, the method comprising:
measuring an initial pressure of at least one of a gas and a fluid bladder disposed within the footwear;
subsequent to placing a load on the footwear by a foot of a user, measuring a resultant load pressure;
calculating a pressure differential between the initial pressure and the resultant load pressure;
converting the pressure differential into a weight measurement; and
transmitting at least one of the pressure differential and the weight measurement to the electronic device.

16. The method of claim 15, wherein measuring the initial pressure comprises:
prior to insertion of the foot into the footwear, converting electrical signals of one or more pressure sensors into an initial weight measurement.

17. The method of claim 15, wherein measuring the resultant load pressure comprises:
subsequent to insertion of the foot into the footwear, converting electrical signals of one or more pressure sensors into a resultant weight measurement.

18. The method of claim 15, further comprising:
providing substantially consistent structural thickness of the at least one of the gas and the fluid bladder by intermittent structural members in communication with an upper and a lower surface of the at least one of the gas and the fluid bladder.

19. The method of claim 15, further comprising:
increasing or decreasing pressure within the at least one of the gas and the fluid bladder.

20. The method of claim 15, further comprising:
measuring at least one of body fat percentage, body water percentage, and steps counted.
